(19) United States
(12) Patent Application Publication
(10) Pub. No.: US 2026/0167846 A1
(43) Pub. Date: Jun. 18, 2026

(54) ARTICLE COMPRISING ADHESIVE AND METHOD OF USE THEREOF

(71) Applicant: Solventum Intellectual Properties Company, Maplewood, MN (US)

(72) Inventors: Audrey A. SHERMAN, Saint Paul, MN (US); Kristin J. GODBEY, Saint Paul, MN (US); Dylan T. COSGROVE, Saint Paul, MN (US); Anne C.F. GOLD, Saint Paul, MN (US); Abhishek SRIVASTAVA, Saint Paul, MN (US); Thomas R. CORRIGAN, Saint Paul, MN (US); Jingyi GUO, Saint Paul, MN (US); Del R. LAWSON, Saint Paul, MN (US); Chad M. HERRLEIN, Saint Paul, MN (US); Graham E. SMITH, Saint Paul, MN (US); Gene P. MCNAMARA, Saint Paul, MN (US)

(73) Assignee: Solventum Intellectual Properties Company, Maplewood, MN (US)

(21) Appl. No.: 19/119,560

(22) PCT Filed: Oct. 9, 2023
(86) PCT No.: PCT/IB2023/060116

§ 371 Date: Apr. 9, 2025

Related U.S. Application Data
(60) Provisional application No. 63/379,195, filed on Oct. 12, 2022.

(51) Int. Cl. C09J 7/20; B32B 7/06; B32B 7/12; C09J 5/00; C09J 7/26

(52) U.S. Cl. CPC C09J 7/203; B32B 7/06; B32B 7/12; C09J 5/00; C09J 7/26; C09J 2203/37; C09J 2301/124; C09J 2301/204; C09J 2301/302; C09J 2301/308; C09J 2400/243

(57) ABSTRACT

An article includes at least one substrate and at least one adhesive member. The at least one adhesive member includes an adhesive layer including an adhesive configured to bond the at least one adhesive member to an external surface. The at least one adhesive member further includes at least one slit extending through the at least one adhesive member. The article further includes at least one tab connected to at least the adhesive layer of the at least one adhesive member and extending from the at least one adhesive member. The at least one tab is configured to receive a pull force from a user. Upon application of the pull force on the at least one tab, the at least one adhesive member at least partially separates adjacent to the at least one slit and releases from each of the at least one substrate and the external surface.

### TECHNICAL FIELD

The present disclosure generally relates to an article including an adhesive and a method of using the article.

### BACKGROUND

Articles that include adhesive layers are generally found in a variety of applications. Such articles may be held against an external surface with the help of the adhesive layer. For example, medical diagnostic devices are typically held against the skin of a user through an adhesive layer. In some cases, the adhesive layer may not be directly accessible to the user if the user wishes to remove the article from the external surface.

Some articles are provided with stretch-release adhesive layers which may be removed by stretching the adhesive layer at certain degree angles without any damage or residue. An example of the stretch-release adhesive layer is provided in WO Patent Application 2021/137,135 (hereinafter referred to as '135 reference). The '135 reference discloses a stretch-release adhesive article with tunable mechanical properties. The stretch-release adhesive article includes a stretchable carrier with one of the major surfaces being a stretch-releasable adhesive surface. The stretchable carrier includes a pattern of slits distributed thereon and configured to deform upon a stretch on the stretchable carrier to elongate the stretchable carrier.

### SUMMARY

In one aspect, an article is described. The article includes at least one substrate. The article further includes at least one adhesive member including a first major surface and a second major surface opposite to the first major surface. The first major surface is connected to the at least one substrate. The at least one adhesive member further includes an adhesive layer forming the second major surface of the at least one adhesive member. The adhesive layer includes an adhesive configured to bond the at least one adhesive member to an external surface. The at least one adhesive member further includes at least one slit extending through the at least one adhesive member from the first major surface to the second major surface. The article further includes at least one tab connected to at least the adhesive layer of the at least one adhesive member and extending from the at least one adhesive member. The at least one tab is configured to receive a pull force from a user. Upon application of the pull force on the at least one tab, the at least one adhesive member at least partially separates adjacent to the at least one slit and releases from each of the at least one substrate and the external surface.

In another aspect, a method of using an article is described. The article includes at least one substrate, at least one adhesive member connected to the at least one substrate, and at least one tab connected to the at least one adhesive member. The method includes adhesively bonding the at least one adhesive layer to an external surface. The method further includes applying a pull force on the at least one tab to cause at least a partial separation of the at least one adhesive member adjacent to at least one slit, thereby releasing the at least one adhesive member from each of the at least one substrate and the external surface.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1A is a schematic top perspective view of an article, according to an embodiment of the present disclosure;

FIG. 1B is a schematic bottom perspective view of the article, according to an embodiment of the present disclosure;

FIG. 2 is a schematic sectional perspective view of the article, according to an embodiment of the present disclosure;

FIG. 3 is a schematic bottom view of the article, according to an embodiment of the present disclosure;

FIG. 4 is a schematic bottom view of at least one adhesive member of the article, according to an embodiment of the present disclosure;

FIG. 5A is a schematic bottom view of a portion of the at least one adhesive member and a tab, according to an embodiment of the present disclosure;

FIG. 5B is a schematic bottom view of the portion of the at least one adhesive member and the tab where a pull force is applied on the tab, according to an embodiment of the present disclosure;

FIG. 6A is a schematic bottom view of the at least one adhesive member and the tab, according to another embodiment of the present disclosure;

FIG. 6B is a schematic bottom view of a portion of the at least one adhesive member and the tab of FIG. 6A where a pull force is applied on the tab, according to an embodiment of the present disclosure;

FIG. 7 is a schematic bottom view of the portion of the at least one adhesive member, according to another embodiment of the present disclosure;

FIG. 8A is a schematic front sectional view of the portion of the at least one adhesive member and the tab, according to an embodiment of the present disclosure;

FIG. 8B is a schematic front sectional view of the portion of the at least one adhesive member and the tab, according to another embodiment of the present disclosure;

FIG. 9A is a schematic front sectional view of the portion of the at least one adhesive member, according to another embodiment of the present disclosure;

FIG. 9B is a schematic sectional side view of the portion of the at least one adhesive member of FIG. 9A, according to an embodiment of the present disclosure;

FIG. 9C is a schematic sectional side view of the portion of the at least one adhesive member of FIG. 9A, according to another embodiment of the present disclosure;

FIG. 10A is a schematic bottom view of the at least one adhesive member, according to another embodiment of the present disclosure;

FIG. 10B is a schematic sectional view of the at least one adhesive member taken along a section line A-A′ shown in FIG. 10A, according to an embodiment of the present disclosure;

FIG. 11 is a schematic bottom view of the at least one adhesive member, according to another embodiment of the present disclosure;

FIG. 12 is a schematic bottom view of the at least one adhesive member, according to yet another embodiment of the present disclosure;

FIG. 13 is a schematic bottom view of the at least one adhesive member, according to another embodiment of the present disclosure;

FIG. 14A is a schematic perspective view of the article, according to another embodiment of the present disclosure;

FIG. 14B is a schematic bottom view of the article of FIG. 14A, according to an embodiment of the present disclosure;

FIG. 15A is a schematic perspective view of the article, according to another embodiment of the present disclosure;

FIG. 15B is a schematic bottom view of the article of FIG. 15A, according to an embodiment of the present disclosure;

FIG. 16 is a schematic perspective view of the article, according to another embodiment of the present disclosure;

FIG. 17 is a schematic bottom view of the article of FIG. 16, according to an embodiment of the present disclosure; and

FIG. 18 is a flow chart illustrating a method of using the article, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As used herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

As used herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein, the term “adhesive” generally refers to polymeric compositions useful to adhere together two adherends. Examples of adhesive may include curable adhesives, heat activated adhesives, pressure sensitive adhesives, or combinations thereof.

As used herein, the term “Layer” generally refers to a planar sheet, film, fabric, or covering of matter formed using only one formulated composition or individual substance.

According to aspects of this disclosure, an article includes at least one substrate. The article further includes at least one adhesive member including a first major surface and a second major surface opposite to the first major surface. The first major surface is connected to the at least one substrate. The at least one adhesive member further includes an adhesive layer forming the second major surface of the at least one adhesive member. The adhesive layer includes an adhesive configured to bond the at least one adhesive member to an external surface. The at least one adhesive member further includes at least one slit extending through the at least one adhesive member from the first major surface to the second major surface. The article further includes at least one tab connected to at least the adhesive layer of the at least one adhesive member and extending from the at least one adhesive member. The at least one tab is configured to receive a pull force from a user. Upon application of the pull force on the at least one tab, the at least one adhesive member at least partially separates adjacent to the at least one slit and releases from each of the at least one substrate and the external surface.

In the article of the present disclosure, the at least one tab is connected to at least the adhesive layer of the at least one adhesive member. Therefore, pulling the at least one tab may result in an application of a force on the at least one adhesive layer. The at least one tab may be accessible to a user of the article. Pulling the at least one tab may enable at least a partial separation of the at least one adhesive member adjacent to the at least one slit, thereby releasing the at least one adhesive member from each of the at least one substrate and the external surface. Thus, the user may be able to conveniently remove the article from the external surface by pulling the at least one tab.

The at least one adhesive member may include a bulk material allowing the at least one adhesive member to be pulled and at least partially separate adjacent to the at least one slit. Further, the at least one slit may allow reduction in a force required to release the at least one adhesive member (e.g., through necking) as compared to conventional adhesive members that include a continuous bulk of adhesive.

Further, as compared to the conventional adhesive members that include a discontinuous pattern of slits, the at least one slit of the present disclosure may be continuous and may extend from the at least one tab until an end of the at least one adhesive member along the first major surface or the second major surface. Further, the conventional adhesive members (with perforations or holes) are pulled as a single continuous body. However, the at least one slit of the present disclosure may allow the at least one adhesive member to be formed as a single, elongated continuous bulk material with a narrow cross-sectional area. This may enable a smaller necking cross-sectional area for the at least one adhesive member upon application of the pull force on the at least one tab, thereby reducing a magnitude of the pull force required for releasing the at least one adhesive member. Therefore, an impact of releasing the at least one adhesive member on both the at least one substrate and the external surface may be minimized.

In some examples, the at least one adhesive member includes a plurality of slits that are substantially parallel to each other. This may divide a necking region of the at least one adhesive member into a plurality of smaller necking regions of reduced cross-sectional area, while the conventional adhesive members include a single necking region, and the entire cross-sectional area of the at least one adhesive member orthogonal to a direction of the pull force is activated or stretched at the same time. Therefore, the at least one adhesive member of the present disclosure with the plurality of slits may further reduce the magnitude of the pull force required for releasing the at least one adhesive member.

Additionally, the plurality of smaller necking regions with reduced cross-sectional area generated in the at least one adhesive member by the plurality of slits may be activated or stretched individually, sequentially, or in parallel, upon application of the pull force on the at least one tab. Further, the at least one tab may include one or more tabs attached to each of the plurality of necking regions to aid in removal of the at least one adhesive member. In some embodiments, the plurality of smaller necking regions with reduced cross-sectional area may result in a lower pull force being required to remove the at least one adhesive member from each of the at least one substrate and the external surface.

It should be noted that the plurality of slits of the at least one adhesive member may not significantly reduce adhesion of the at least one adhesive member to the external surface (such as the skin of a user) while facilitating release of the at least one adhesive member from each of the at least one substrate and the external surface without any significant damage or residue. Moreover, the at least one slit may allow transmission of moisture through the at least one adhesive member in case where the external surface is the skin of the user, thereby improving a permeability and a breathability of the at least one adhesive member.

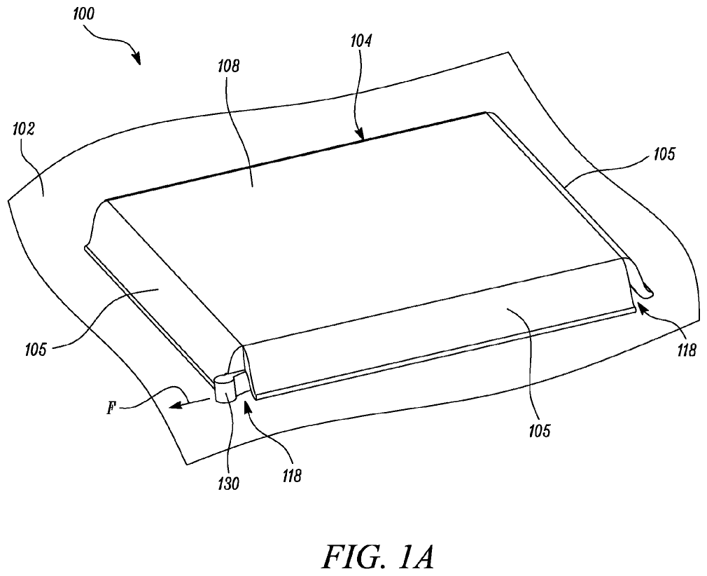

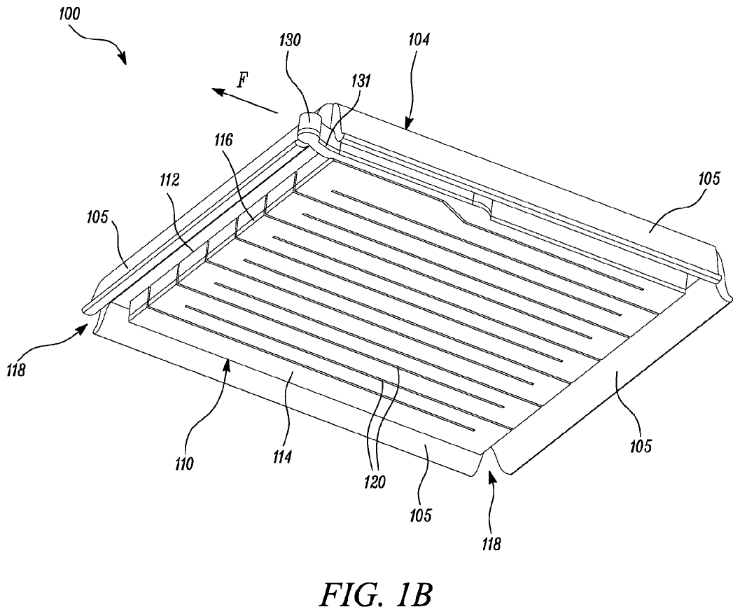

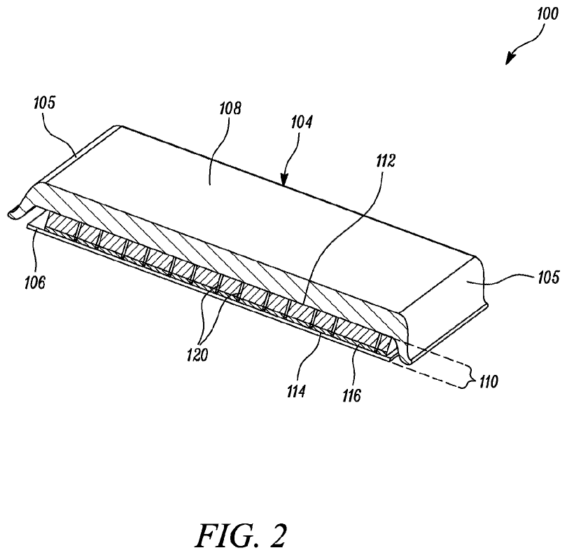

Referring now to Figures, FIG. 1A is a schematic top perspective view of an article 100. The article 100 is attached to an external surface 102. FIG. 1B is a schematic bottom perspective view of the article 100. FIG. 2 is a schematic sectional perspective view of the article 100. Referring now to FIGS. 1A-2, the article 100 includes at least one substrate 104. The article 100 further includes at least one adhesive member 110 (shown in FIGS. 1B and 2). The article 100 is attached to the external surface 102 via the adhesive member 110. The term “at least one substrate 104” is interchangeably referred to hereinafter as “the substrate 104”. The term “at least one adhesive member 110” is interchangeably referred to hereinafter as “the adhesive member 110”.

In some examples, the at least one substrate 104 includes a main portion 108 connected with the at least one adhesive member 110. In the illustrated example of FIGS. 1A-2, the main portion 108 is rectangular (i.e., polygonal). In some other examples, the main portion 108 may be circular, oval, or elliptical. It should be understood that the main portion 108 may be of any shape and geometry based on application requirements, including irregular shapes.

In some examples, the substrate 104 further includes one or more side portions 105 extending from the main portion 108. In some examples, the one or more side portions 105 at least partially surround the at least one adhesive member 110. In the illustrated example of FIGS. 1A-2, the one or more side portions 105 includes a plurality of side portions 105 spaced apart from each other. In some examples, the plurality of side portions 105 may prevent external agents, such as dirt, from reaching the adhesive member 110.

In some examples, the at least one substrate 104 includes at least one cutout 118. Specifically, the at least one cutout 118 includes a plurality of cutouts 118 separating the plurality of side portions 105 from each other. In some examples, each cutout 118 from the plurality of cutouts 118 is disposed between corresponding adjacent pair of side portions 105 from the plurality of side portions 105.

In some examples, the substrate 104 may be a backing for the adhesive member 110. In some examples, the substrate 104 may be a part of a medical diagnostic device and the external surface 102 may be skin of a user. For example, the medical diagnostic device may be attached to the skin of the user through the adhesive member 110. In some examples, the medical diagnostic device may include one or more sensors mounted thereon.

As shown in FIGS. 1B and 1C, the at least one adhesive member 110 includes a first major surface 112 and a second major surface 114 opposite to the first major surface 112. The first major surface 112 is connected to the at least one substrate 104. The at least one adhesive member 110 further includes an adhesive layer 116 forming the second major surface 114 of the at least one adhesive member 110.

The adhesive layer 116 includes an adhesive configured to bond the at least one adhesive member 110 to the external surface 102 (shown in FIG. 1A). In some examples, the adhesive is a pressure sensitive adhesive or a stretch-release adhesive. In some examples, the article 100 further includes a release liner 106 (shown in FIG. 2) releasably attached to the adhesive layer 116. In some examples, the release liner 106 is removed prior to bonding of the adhesive layer 116 with the external surface 102 (shown in FIG. 1A). The release liner 106 is not shown in FIGS. 1A and 1B.

The at least one adhesive member 110 further includes at least one slit 120 extending through the at least one adhesive member 110 from the first major surface 112 to the second major surface 114. The at least one slit 120 is therefore a through-slit. In some examples, the at least one slit 120 may further extend along the first major surface 112 or the second major surface 114. In some examples, the at least one slit 120 may be disposed in a predetermined pattern along the first major surface 112 and the second major surface 114. The term “at least one slit 120” is interchangeably referred to hereinafter as “the slit 120”.

In some examples, the slit 120 may be generated by cutting through the adhesive member 110 by a variety of known methods. Specifically, the slit 120 may be generated by cutting through a continuous region of the adhesive member 110 in the predetermined pattern, such that the predetermined pattern may impart specific mechanical properties to the adhesive member 110. Such mechanical properties may also differ in different directions. Alternatively, the adhesive member 110 may dispensed directly from a robotic platform in the predetermined pattern, e.g., in the form of elongate portions. It should be understood that the adhesive member 110 may be formed in different shapes/geometries and the slit 120 may be formed in different shapes/patterns based on application requirements.

As shown in FIGS. 1A and 1B, the article 100 further includes at least one tab 130 connected to at least the adhesive layer 116 of the at least one adhesive member 110 and extending from the at least one adhesive member 110. In some examples, the at least one cutout 118 is configured to at least partially receive the at least one tab 130 therethrough. In some examples, the at least one tab 130 may be made from a different material as that of the adhesive member 110. In some other examples, the at least one tab 130 may be made from same material as that of the adhesive member 110. The term “at least one tab 130” is interchangeably referred to hereinafter as “the tab 130”.

In some examples, the at least one tab 130 and the at least one adhesive member 110 have the same layered construction. In other words, the at least one tab 130 and the at least one adhesive member 110 include the same layers (i.e., similar composition) having the same respective thicknesses. In some examples, the at least one tab 130 includes an adhesive layer 131. In some examples, the adhesive layer 131 includes a pressure sensitive adhesive or a stretch release adhesive. In some examples, the adhesive layer 131 may include an adhesive similar to the adhesive of the adhesive layer 116. Alternatively, in some other examples, the at least one tab 130 may be non-tacky.

The at least one tab 130 is configured to receive a pull force F from the user. Upon application of the pull force F on the at least one tab 130, the at least one adhesive member 110 at least partially separates adjacent to the at least one slit 120 and releases from each of the at least one substrate 104 and the external surface 102. Thus, the user may be able to release the substrate 104 from the external surface 102 by pulling the tab 130.

In some examples, the at least one tab 130 may be tacky. For example, the at least one tab 130 may be an extension of the at least one adhesive member 110. In such cases, the at least one tab 130 may be grasped and pulled by the user to stretch the at least one adhesive member 110 for releasing the substrate 104 from the external surface 102. In some examples, the at least one tab 130 may include those described in U.S. Pat. No. 9,587,146 (Sherman et al.) and U.S. Pat. No. 9,228,117 (Sherman et al.), the disclosures of which are incorporated by reference in their entirety.

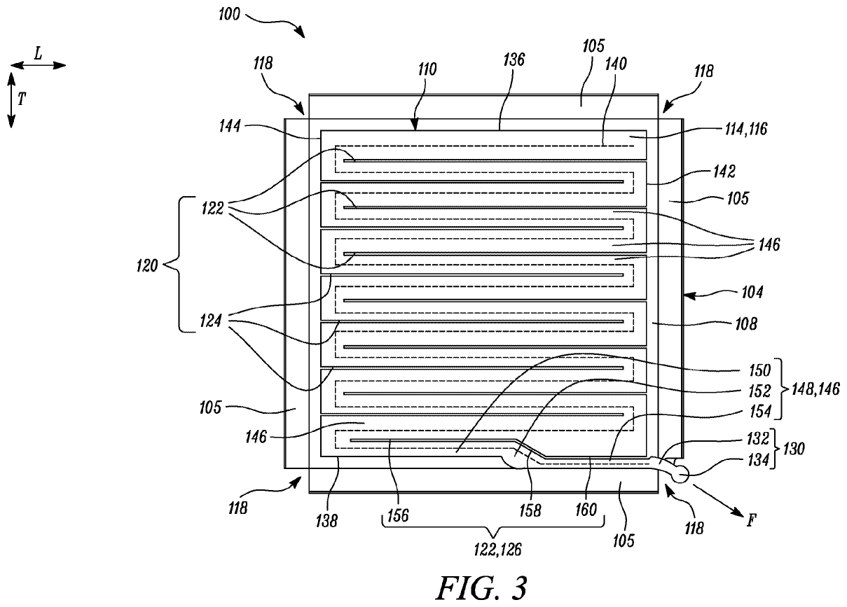

FIG. 3 is a schematic bottom view of the article 100. In some examples, the at least one adhesive member 110 further includes a first longitudinal edge 136 extending along a longitudinal axis L, a second longitudinal edge 138 opposing the first longitudinal edge 136 and extending at least partially along the longitudinal axis L, a first transverse edge 142 extending between the first longitudinal edge 136 and the second longitudinal edge 138 along a transverse axis T orthogonal to the longitudinal axis L, and a second transverse edge 144 opposing the first transverse edge 142 and extending between the first longitudinal edge 136 and the second longitudinal edge 138 along the transverse axis T.

In some examples, the at least one slit 120 includes a plurality of slits 120 spaced apart from each other with respect to the transverse axis T and extending at least partially along the longitudinal axis L. In some examples, the plurality of slits 120 includes a set of first slits 122 and a set of second slits 124 alternating with the set of first slits 122. In some examples, each first slit 122 from the set of first slits 122 extends from the first transverse edge 142 partially towards the second transverse edge 144, such that each first slit 122 is spaced apart from the second transverse edge 144. In some examples, each second slit 124 from the set of second slits 124 extends from the second transverse edge 144 partially towards the first transverse edge 142, such that each second slit 124 is spaced apart from the first transverse edge 142. It should be understood that the set of first slits 122 and the set of second slits 124 may include any number of first slits 122 and second slits 124, respectively.

In some examples, the at least one adhesive member 110 includes a plurality of elongate portions 146 extending between the first transverse edge 142 and the second transverse edge 144 at least partially along the longitudinal axis L. In some examples, adjacent elongate portions 146 from the plurality of elongate portions 146 are at least partially separated from each other by a corresponding first slit 122 from the set of first slits 122 or a corresponding second slit 124 from the set of second slits 124. In some examples, the adjacent elongate portions 146 are connected to each other at the first transverse edge 142 or the second transverse edge 144, such that the plurality of elongate portions 146 together form a serpentine pattern 140. Thus, the set of first slits 122 and the set of second slits 124 may allow the at least one adhesive member 110 to be formed as a single, elongated continuous bulk material with a narrow cross-sectional area.

In some examples, the plurality of elongate portions 146 includes an end elongate portion 148 disposed at the second longitudinal edge 138. In some examples, the set of first slits 122 includes an end first slit 126 disposed proximal to the second longitudinal edge 138 and at least partially separating the end elongate portion 148 from the adjacent elongate portion 146.

In some examples, the end elongate portion 148 includes a first elongate portion 150 connected to the adjacent elongate portion 146 and extending along the longitudinal axis L, a second elongate portion 152 extending from and inclined obliquely to the first elongate portion 150, and a third elongate portion 154 extending from the second elongate portion 152 along the longitudinal axis L.

In some examples, the end first slit 126 includes a first slit portion 156 extending from the first transverse edge 142 along the longitudinal axis L, a second slit portion 158 extending from and inclined obliquely to the first slit portion 156, and a third slit portion 160 extending from the second slit portion 158 along the longitudinal axis L partially towards the second transverse edge 144.

In some examples, the at least one tab 130 is connected to the end elongate portion 148 and disposed adjacent to the end first slit 126. Specifically, the at least one tab 130 extends from the third elongate portion 154. In some examples, the at least one tab 130 includes a curved portion 132 extending from the at least one adhesive member 110 and a disc-shaped portion 134 connected to the curved portion 132 distal to the at least one adhesive member 110.

The tab 130 is configured to receive the pull force F from the user. Upon application of the pull force F by the user on the tab 130, the adhesive member 110 at least partially separates adjacent to the plurality of slits 120 and releases from each of the substrate 104 and the external surface 102 (shown in FIG. 1), e.g., through necking. In other words, upon application of the pull force F, the serpentine pattern 140 of the at least one adhesive member 110 unwinds (e.g., like a ribbon) along the plurality of slits 120 and gets released from each of the substrate 104 and the external surface 102 (shown in FIG. 1). Since the at least one adhesive member 110 of the present disclosure is formed as a single, elongated continuous bulk material with a narrow cross-sectional area orthogonal to a direction of the pull force F, a magnitude of the pull force F required for releasing the at least one adhesive member 110 may be reduced as compared to conventional adhesive members (with perforations or holes) that are pulled as a single continuous body.

In some examples, upon application of the pull force F by the user on the tab 130, the adhesive member 110 may slide out from the at least one cutout 118, thereby releasing the substrate 104 from the external surface 102 (shown in FIG. 1). Alternatively, upon application of the pull force F by the user on the tab 130, the adhesive member 110 may slide out underneath the one or more side portions 105, thereby releasing the substrate 104 from the external surface 102 (shown in FIG. 1).

In some examples, the adhesive member 110 may include a bulk material allowing the adhesive member 110 and the adhesive layer 116 to receive a pull force through the tab 130 without breakage. In other words, the adhesive member 110 is strong enough to withstand the pull forces that are applied to the adhesive member 110. For example, suitable material may be chosen for the adhesive member 110 that provides structural strength to the adhesive member 110 based on chemical properties or intrinsic material properties. It should be understood that any suitable material may be utilized for the adhesive member 110 that provides sufficient strength to the adhesive member 110.

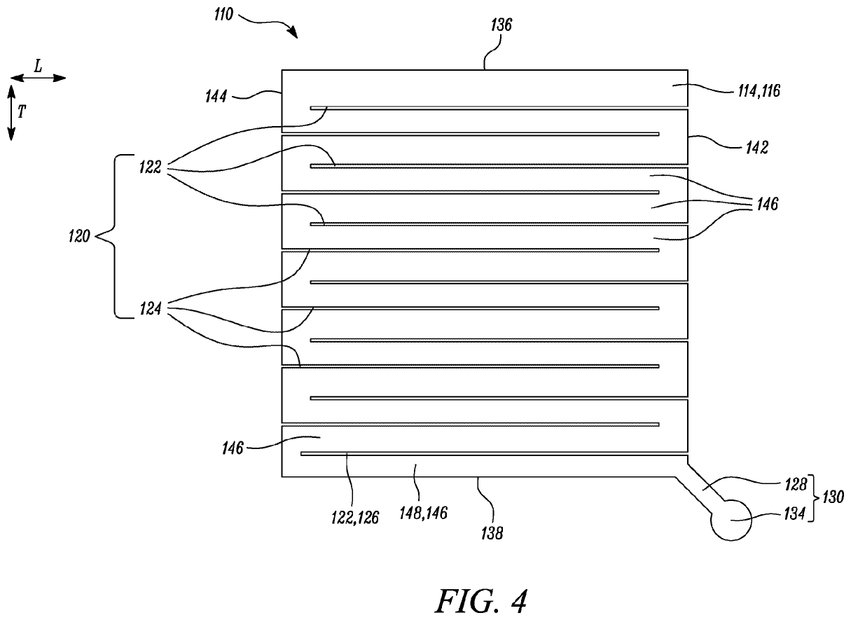

FIG. 4 is a schematic bottom view of the at least one adhesive member 110. In the illustrated example of FIG. 4, each of the end first slit 126 and the end elongate portion 148 extends along the longitudinal axis L. In some examples, the at least one tab 130 includes a linear portion 128 extending from the at least one adhesive member 110 and the disc-shaped portion 134 connected to the linear portion 128 distal to the at least one adhesive member 110.

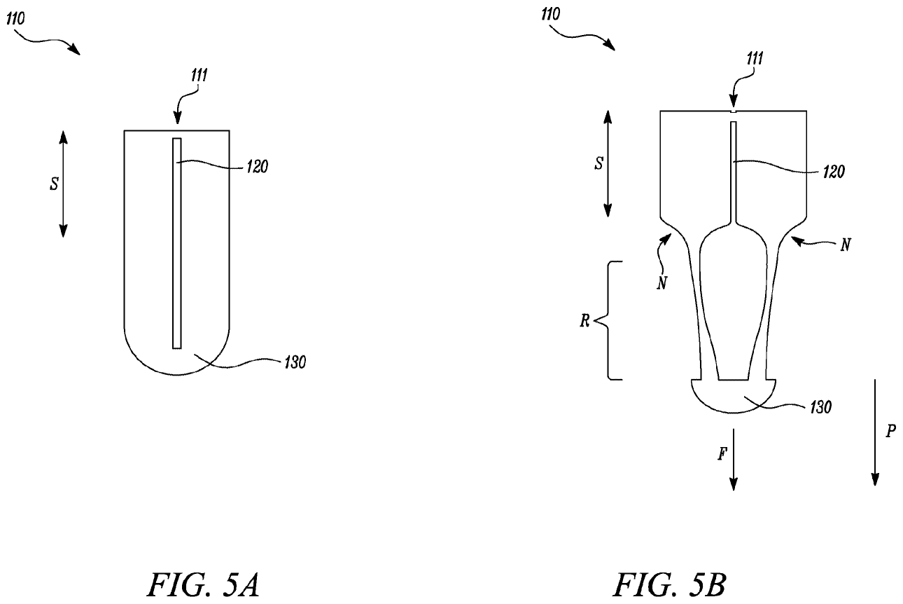

FIG. 5A is a schematic bottom view of a portion of the adhesive member 110 and the tab 130. In the illustrated example of FIG. 5A, a single slit 120 is shown for the purpose of illustration. FIG. 5B is a schematic bottom view of the portion of the adhesive member 110 and the tab 130 where the pull force F is applied on the tab 130. In some examples, the slit 120 extends along a slit axis S and the pull force F is applied along a pull direction P. In the illustrated example of FIG. 5B, the pull direction P is substantially parallel to the slit axis S of the slit 120.

Referring now to FIGS. 5A and 5B, in some examples, the adhesive member 110 releases from each of the substrate 104 (shown in FIGS. 1A-3) and the external surface 102 (shown in FIG. 1A) by necking in response to the application of the pull force F on the tab 130. Specifically, the adhesive member 110 necks down to form necking regions N in response to the application of the pull force F on the tab 130 before being fully released, as indicated by a released region R. Thus, the adhesive member 110 may first stretch to become narrow before the adhesive member 110 is fully released.

The adhesive member 110 of the present disclosure includes the at least one slit 120 that extends from the at least one tab 130 until an end 111 or proximal to the end 111 of the at least one adhesive member 110. Further, the at least one slit 120 may enable the at least one adhesive member 110 to be released by forming multiple smaller necking regions N as compared to conventional adhesive members that include a single necking region and the entire cross-sectional area of the adhesive member orthogonal to a direction of a pull force is activated or stretched at the same time. Therefore, a magnitude of the pull force F required to release the adhesive member 110 may be significantly lower than the case where the adhesive member includes a bulk material that is continuous or with discontinuous slits, resulting in lower damage to bonded surfaces.

In some examples, the magnitude of the pull force F required to release the adhesive member 110 may depend upon a width of the adhesive member 110. Thus, reducing the width of the adhesive member 110 may reduce the pull force F required to release the adhesive member 110. Further, the magnitude of the pull force F required to release the adhesive member 110 may increase with increase in the width of the adhesive member 110. Thus, if a wider adhesive member 110 may be provided, multiple slits 120 may be used to allow generation of multiple necking regions, thereby reducing the magnitude of the pull force F required to release the adhesive member 110.

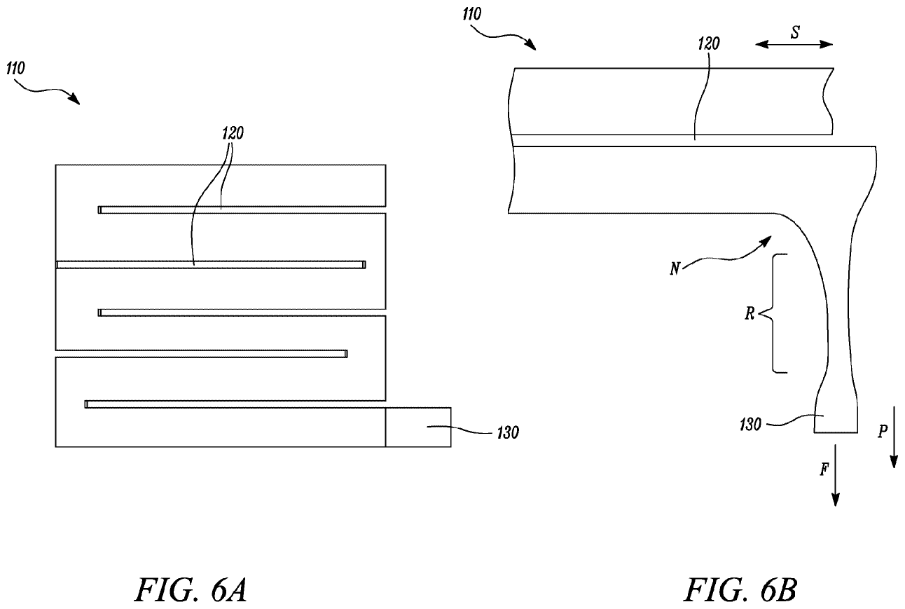

FIG. 6A is a schematic bottom view of the adhesive member 110 and the tab 130, according to another embodiment of the present disclosure. FIG. 6B is a schematic bottom view of a portion of the adhesive member 110 and the tab 130 where the pull force F is applied on the tab 130. Specifically, the pull direction P of the pull force F is inclined to the slit axis S of the slit 120.

Referring now to FIGS. 6A and 6B, the magnitude of the pull force F required to release the adhesive member 110 when the pull direction P of the pull force F is inclined to the slit axis S of the slit 120 is still lower than the case where the adhesive member 110 includes a continuous bulk of adhesive. In some examples, the adhesive member 110 releases from each of the substrate 104 (shown in FIGS. 1A-3) and the external surface 102 (shown in FIG. 1A) by necking in response to the application of the pull force F on the tab 130. Specifically, the adhesive member 110 necks down to form the necking region N in response to the application of the pull force F on the tab 130 before being fully released, as indicated by the released region R. Reduction in the magnitude of the pull force F may also be attributed to the presence of the slit 120, resulting in lower damage to bonded surfaces.

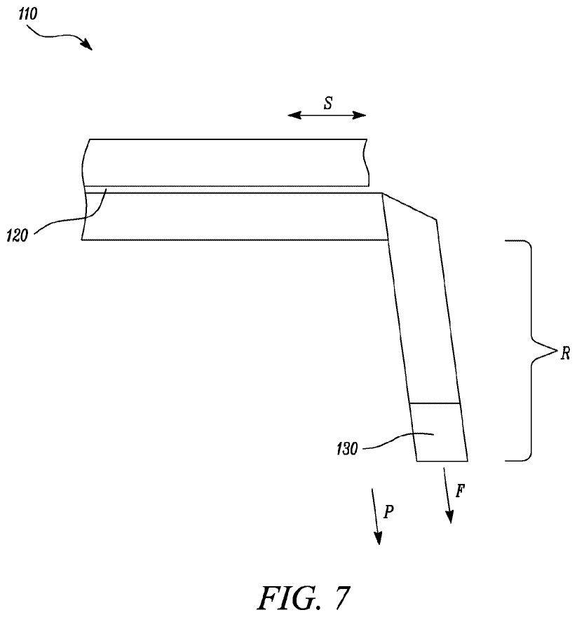

FIG. 7 is a schematic bottom view of a portion of the adhesive member 110, according to another embodiment of the present disclosure. In the illustrated example of FIG. 7, a single slit 120 is shown for the purpose of illustration. Further, the pull direction P of the pull force F is inclined to the slit axis S of the slit 120.

In some examples, the adhesive member 110 releases from each of the substrate 104 (shown in FIGS. 1A-3) and the external surface 102 (shown in FIG. 1A) by rolling. In some examples, when the pull direction P of the pull force F is not aligned with the slit axis S of the slit 120, it may be possible that the adhesive member 110 rolls over itself and releases from the substrate 104 (shown in FIGS. 1A-3) and the external surface 102 (shown in FIG. 1A) before the adhesive member 110 experiences sufficient forces to cause necking. This release mechanism may depend upon a function of a bond strength to the bonded surface and a modulus of elasticity of the adhesive member 110. The magnitude of the pull force F required to release the adhesive member 110 with the slit 120 is still lower than the case where the adhesive member 110 includes a continuous bulk of adhesive.

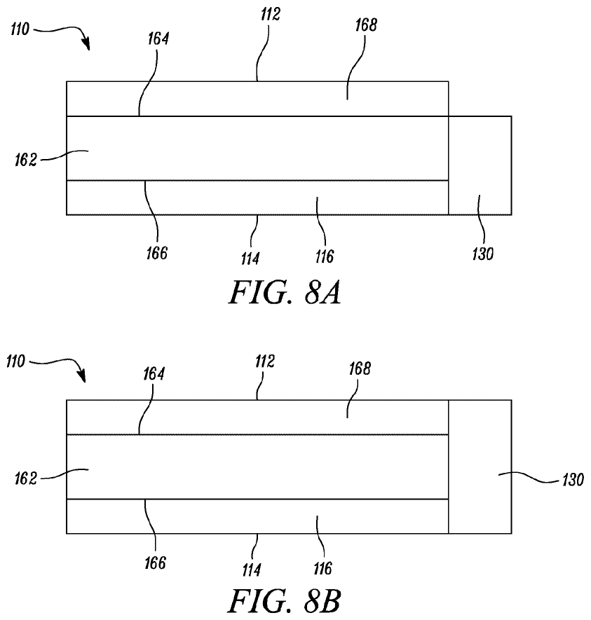

FIG. 8A is a schematic front sectional view of a portion of the adhesive member 110 and the tab 130. In the illustrated embodiment of FIG. 8A, the at least one adhesive member 110 further includes a core layer 162 including a first core major surface 164 facing the at least one substate 104 (shown in FIGS. 1A-3) and a second core major surface 166 opposing the first core major surface 164.

In some examples, the adhesive layer 116 is disposed on the second core major surface 166. In the illustrated example of FIG. 8A, the tab 130 is connected to the adhesive layer 116 and the core layer 162 of the adhesive member 110 and extends from the adhesive member 110. In some examples, the at least one adhesive member 110 further includes another adhesive layer 168 disposed on the first core major surface 164 and bonding the at least one adhesive member 110 to the at least one substrate 104 (shown in FIGS. 1A-3). Specifically, the another adhesive layer 168 forms the first major surface 112 of the adhesive member 110.

In some examples, the core layer 162 includes a foam. However, it should be understood that any other suitable material may also be chosen for the core layer 162. In some examples, the core layer 162 may include a thin film layer. In such cases, the thin film layer may match a necking behavior of the adhesive member 110 or may sustain the forces in the pull direction P (shown in FIGS. 5A-7) while not limiting the necking behavior of the adhesive member 110.

In some examples, the core layer 162 may increase a structural strength of the adhesive member 110 allowing the adhesive member 110 to withstand the pull forces applied onto the adhesive member 110 in order to release the adhesive member 110 from each of the substrate 104 (shown in FIGS. 1A-3) and the external surface 102 (shown in FIG. 1A).

FIG. 8B is a schematic front sectional view of a portion of the adhesive member 110 and the tab 130. In the illustrated example of FIG. 8C, the at least one tab 130 is further connected to the core layer 162 and the another adhesive layer 168. Thus, the tab 130 may transfer the pull force F to the core layer 162 as well as the another adhesive layer 168 for releasing the adhesive member 110 from each of the substrate 104 (shown in FIGS. 1A-3) and the external surface 102 (shown in FIG. 1A).

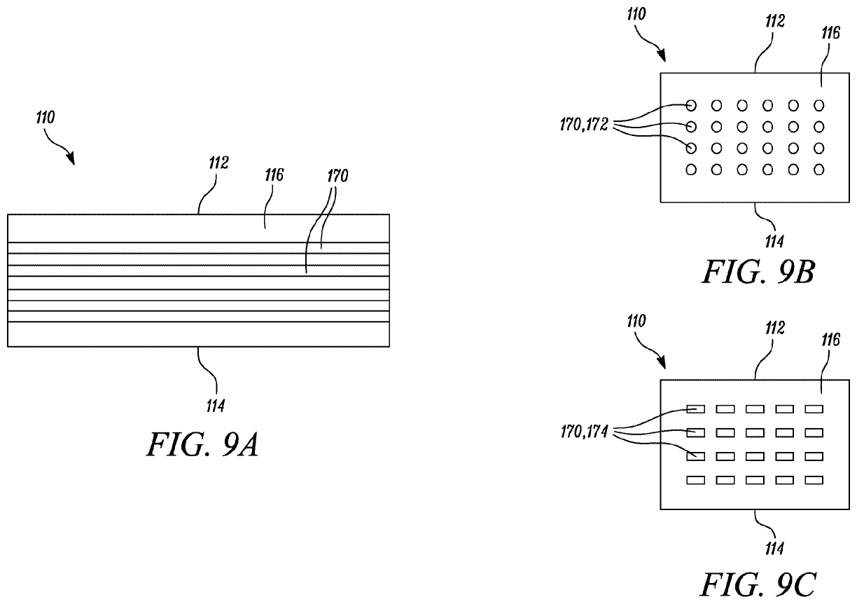

FIG. 9A is a schematic front sectional view of a portion of the adhesive member 110, according to another embodiment of the present disclosure. In the illustrated example of FIG. 9A, the tab 130 is not shown for the purpose of illustration. Further, the adhesive layer 116 further forms the first major surface 112 of the at least one adhesive member 110 and is bonded to the at least one substrate 104 (shown in FIGS. 1A-3).

In some examples, the adhesive layer 116 further includes a plurality of strengthening elements 170 embedded within the adhesive. The plurality of strengthening elements 170 may increase the structural strength of the adhesive member 110 allowing the adhesive member 110 to withstand the pull forces applied on the adhesive member 110. The plurality of strengthening elements 170 may enable the adhesive member 110 to sustain the pull forces applied on the adhesive member 110 while not limiting the necking behavior of the adhesive member 110.

FIG. 9B is a schematic sectional side view of the portion of the adhesive member 110. In the illustrated example of FIG. 9B, the plurality of strengthening elements 170 include cylindrical members 172. FIG. 9C is a schematic sectional side view of the portion of the adhesive member 110, according to another embodiment of the present disclosure. In the illustrated example of FIG. 9C, the plurality of strengthening elements 170 include flat members 174.

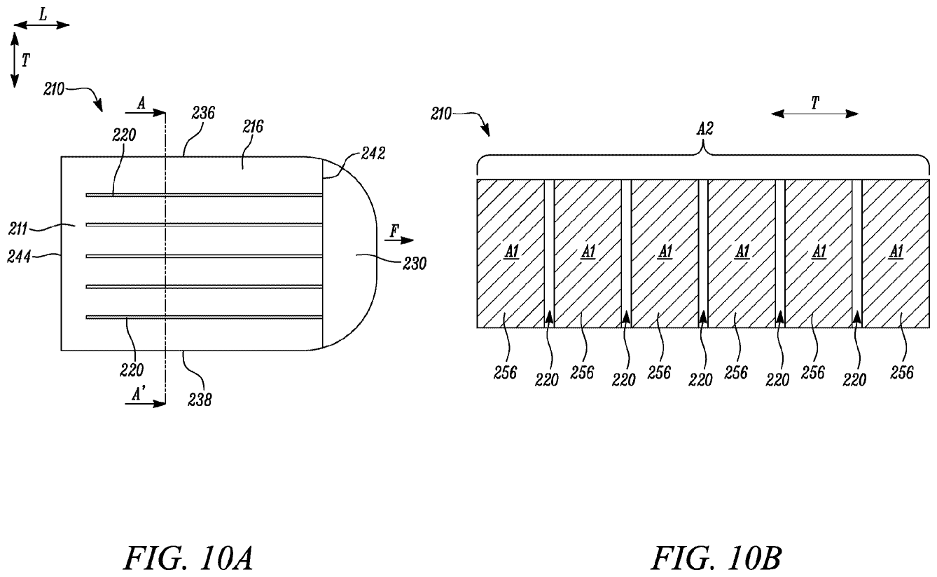

FIG. 10A is a schematic bottom view of at least one adhesive member 210. In some examples, the adhesive member 210 may be functionally equivalent to the adhesive member 110 of FIGS. 1A-9C. In some examples, the at least one adhesive member 210 further includes a first longitudinal edge 236 extending along the longitudinal axis L, a second longitudinal edge 238 opposing the first longitudinal edge 236 and extending at least partially along the longitudinal axis L, a first transverse edge 242 extending between the first longitudinal edge 236 and the second longitudinal edge 238 along the transverse axis T orthogonal to the longitudinal axis L, and a second transverse edge 244 opposing the first transverse edge 242 and extending between the first longitudinal edge 236 and the second longitudinal edge 238 along the transverse axis T.

In some examples, the at least one adhesive member 210 includes at least one slit 220 extending through the at least one adhesive member 210. The at least one slit 220 includes a plurality of slits 220 spaced apart from each other with respect to the transverse axis T and extending at least partially along the longitudinal axis L. In the illustrated example of FIG. 10A, each slit 220 from the plurality of slits 220 extends from the first transverse edge 242 partially towards the second transverse edge 244, such that each slit 220 is spaced apart from the second transverse edge 244. As compared to the conventional adhesive members that include a discontinuous pattern of slits, the plurality of slits 220 of the present disclosure may be continuous and may substantially extend from the first transverse edge 242 towards the second transverse edge 244.

In the illustrated example of FIG. 10A, the plurality of slits 220 are spaced apart from the second transverse edge 244 forming an un-slit portion, i.e., a bridge portion 211. The bridge portion 211 may be a portion of the at least one adhesive member 210 small enough not to affect a necking behavior of the at least one adhesive member 210. Further, in some examples, the bridge portion 211 may improve a handling of the at least one adhesive member 210 during production.

In some examples, at least one tab 230 is connected to at least an adhesive layer 216 of the at least one adhesive member 210 and extends from the at least one adhesive member 210. In some examples, the at least one tab 230 extends from the first transverse edge 242 and is disposed adjacent to the plurality of slits 220. In some examples, the at least one tab 230 tapers away from the at least one adhesive member 210 and is D-shaped. Specifically, the at least one tab 230 tapers away from the first transverse edge 242 of the adhesive member 210. Alternatively, the at least one tab 230 may be connected to the second transverse edge 244 of the adhesive member 210 and may taper away from the second transverse edge 244.

The at least one tab 230 is configured to receive the pull force F from the user. Upon application of the pull force F on the at least one tab 230, the at least one adhesive member 210 releases from each of the at least one substrate 104 (shown in FIGS. 1A-3) and the external surface 102 (shown in FIG. 1A). In some examples, the plurality of slits 220 are parallel to each other. This may divide a necking region of the at least one adhesive member 210 into multiple smaller necking regions of reduced cross-sectional area. Thus, the at least one adhesive member 210 may reduce the magnitude of the pull force F required for releasing the at least one adhesive member 210.

FIG. 10B is a schematic sectional view of the at least one adhesive member 210 taken along a section line A-A′ shown in FIG. 10A. Referring now to FIGS. 10A and 10B, in some examples, the at least one adhesive member 210 includes a plurality of adhesive regions 256 disposed adjacent to the at least one slit 220. Specifically, the plurality of adhesive regions 256 are separated by the plurality of slits 220. In other words, the plurality of slits 220 divide the at least one adhesive member 210 into individual adhesive regions 256. In some examples, each of the plurality of adhesive regions 256 has a cross-sectional area A1 less than a total cross-sectional area A2 of the at least one adhesive member 210. In some examples, the total cross-sectional area A2 of the at least one adhesive member 210 is equal to a sum of the cross-sectional area A1 of individual adhesive regions 256 and a cross-sectional area of the plurality of slits 220.

In some examples, the plurality of slits 220 divide the at least one adhesive member 210 into multiple small necking regions (corresponding to the plurality of adhesive regions 256) of reduced cross-sectional area. In some examples, the plurality of smaller necking regions with reduced cross-sectional area may result in a lower pull force F being required to remove the at least one adhesive member 210 from each of the at least one substrate 104 (shown in FIGS. 1A-3) and the external surface 102 (shown in FIG. 1A) as compared to conventional adhesive members that include a single necking region where an entire cross-sectional area of the adhesive member is activated or stretched at the same time.

In some examples, the plurality of adhesive regions 256 may be activated or stretched individually, sequentially, or in parallel, upon application of the pull force F on the at least one tab 230. In some examples, the at least one tab 230 may include one or more tabs (not shown) attached to each of the plurality of adhesive regions 256 to aid in removal of the at least one adhesive member 210.

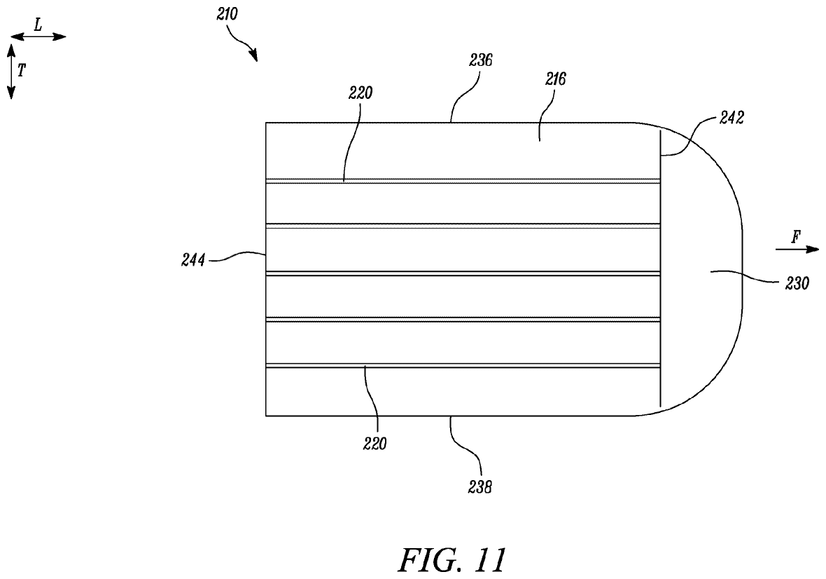

FIG. 11 is a schematic bottom view of at least one adhesive member 210, according to another embodiment of the present disclosure. In the illustrated embodiment of FIG. 11, each slit 220 from the plurality of slits 220 extends from the first transverse edge 242 to the second transverse edge 244. In some examples, the at least one tab 230 extends from the first transverse edge 242 and is disposed adjacent to the plurality of slits 220. In some examples, the at least one tab 230 tapers away from the at least one adhesive member 210 and is D-shaped. Specifically, the at least one tab 230 tapers away from the first transverse edge 242 of the adhesive member 210. Alternatively, the at least one tab 230 may be connected to the second transverse edge 244 of the adhesive member 210 and may taper away from the second transverse edge 244.

The at least one tab 230 is configured to receive the pull force F from the user. Upon application of the pull force F on the at least one tab 230, the at least one adhesive member 210 releases from each of the at least one substrate 104 (shown in FIGS. 1A-3) and the external surface 102 (shown in FIG. 1A).

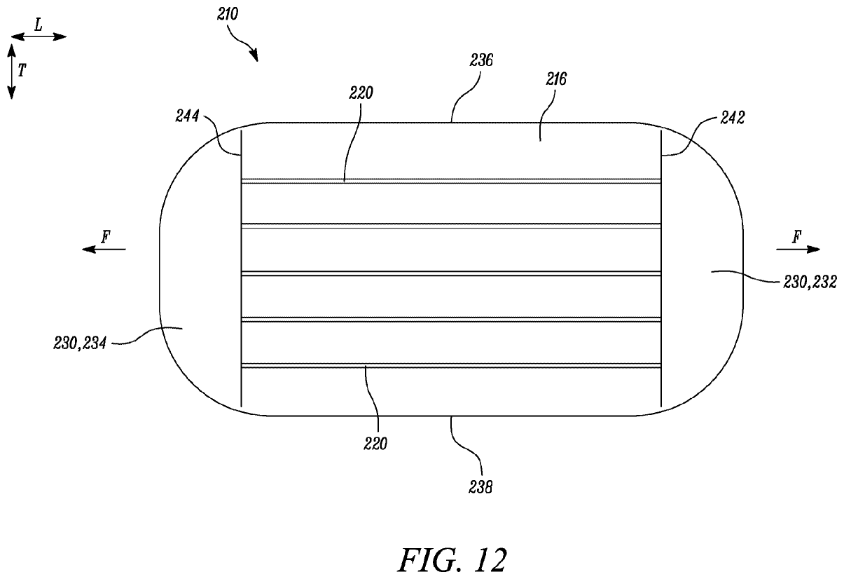

FIG. 12 is a schematic bottom view of at least one adhesive member 210, according to yet another embodiment of the present disclosure. In the illustrated embodiment of FIG. 12, each slit 220 from the plurality of slits 220 extends from the first transverse edge 242 to the second transverse edge 244. In some examples, the at least one tab 230 includes a first tab 232 extending from the first transverse edge 242 and is disposed adjacent to the plurality of slits 220. In some examples, the at least one tab further includes a second tab 234 extending from the second transverse edge 244 and disposed adjacent to the plurality of slits 220. In some examples, each of the first tab 232 and the second tab 234 tapers away from the respective first transverse edge 242 and the second transverse edge 244 and is D-shaped.

In some examples, both the first tab 232 and the second tab 234 are configured to receive the pull force F from the user in opposing directions. Upon application of the pull force F on the first tab 232 and the second tab 234 in opposing directions, the at least one adhesive member 210 releases from each of the at least one substrate 104 (shown in FIGS. 1A-3) and the external surface 102 (shown in FIG. 1A).

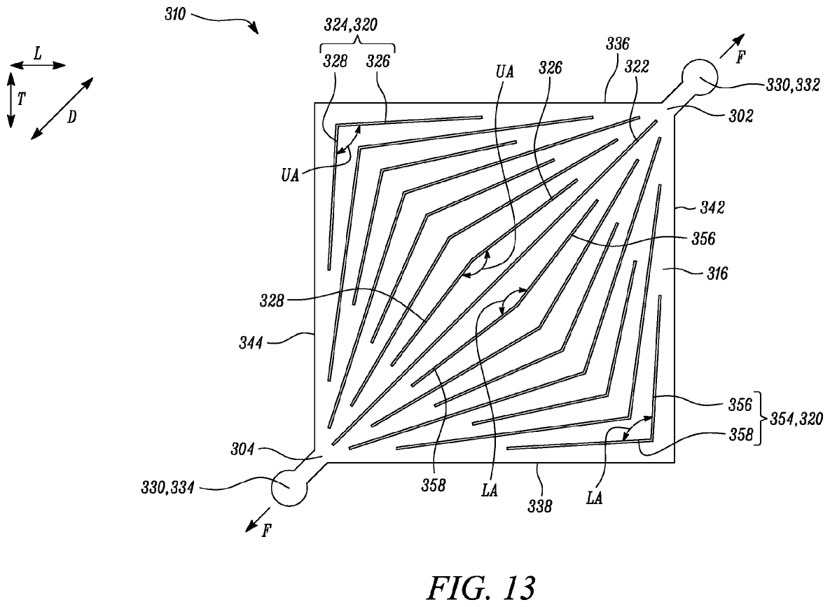

FIG. 13 is a schematic bottom view of at least one adhesive member 310. The at least one adhesive member 310 is functionally equivalent to the at least one adhesive member 110 of FIGS. 1A-9C. In some examples, the at least one adhesive member 310 further includes a first longitudinal edge 336 extending along the longitudinal axis L, a second longitudinal edge 338 opposing the first longitudinal edge 336 and extending at least partially along the longitudinal axis L, a first transverse edge 342 extending between the first longitudinal edge 336 and the second longitudinal edge 338 along the transverse axis T orthogonal to the longitudinal axis L, and a second transverse edge 344 opposing the first transverse edge 342 and extending between the first longitudinal edge 336 and the second longitudinal edge 338 along the transverse axis T.

In some examples, the at least one adhesive member 310 includes at least one slit 320 extending through the at least one adhesive member 310. In some examples, the at least one slit 320 includes a plurality of slits 320 spaced apart from each other. In some examples, the plurality of slits 320 includes a central slit 322 extending along a diagonal axis D of the at least one adhesive member 310. In some examples, the diagonal axis D extends from a first intersection 302 between the first longitudinal edge 336 and the first transverse edge 342 and a second intersection 304 between the second longitudinal edge 338 and the second transverse edge 344.

In some examples, the plurality of slits 320 further includes a set of upper slits 324 disposed between the central slit 322 and the first longitudinal edge 336. In some examples, each upper slit 324 from the set of upper slits 324 includes a first linear portion 326 and a second linear portion 328 inclined to the first linear portion 326 by an upper inclination angle UA.

In some examples, the upper inclination angle UA of each upper slit 324 is less than the upper inclination angle UA of the adjacent upper slit 324 that is located closer to the central slit 322, such that the upper inclination angle UA of the upper slit 324 disposed proximal to the central slit 322 has a maximum value and the upper inclination angle UA of the upper slit 324 disposed proximal to the first longitudinal edge 336 has a minimum value.

In some examples, the plurality of slits 320 further includes a set of lower slits 354 disposed between the central slit 322 and the second longitudinal edge 338. In some examples, each lower slit 354 from the set of lower slits 354 includes a first linear portion 356 and a second linear portion 358 inclined to the first linear portion 356 by a lower inclination angle LA.

In some examples, the lower inclination angle LA of each lower slit 354 is less than the lower inclination angle LA of the adjacent lower slit 354 that is located closer to the central slit 322, such that the lower inclination angle LA of the lower slit 354 disposed proximal to the central slit 322 has a maximum value and the lower inclination angle LA of the lower slit 354 disposed proximal to the second longitudinal edge 338 has a minimum value.

In some examples, at least one tab 330 is connected to at least an adhesive layer 316 of the at least one adhesive member 310 and extends from the at least one adhesive member 310. In some examples, the at least one tab 330 includes a first tab 332 extending from the first intersection 302 along the diagonal axis D and a second tab 334 extending from the second intersection 304 along the diagonal axis D.

In some examples, both the first tab 332 and the second tab 334 are configured to receive the pull force F from the user in opposing directions. Upon application of the pull force F on the first tab 332 and the second tab 334 in opposing directions, the at least one adhesive member 310 releases from each of the at least one substrate 104 (shown in FIGS. 1A-3) and the external surface 102 (shown in FIG. 1A).

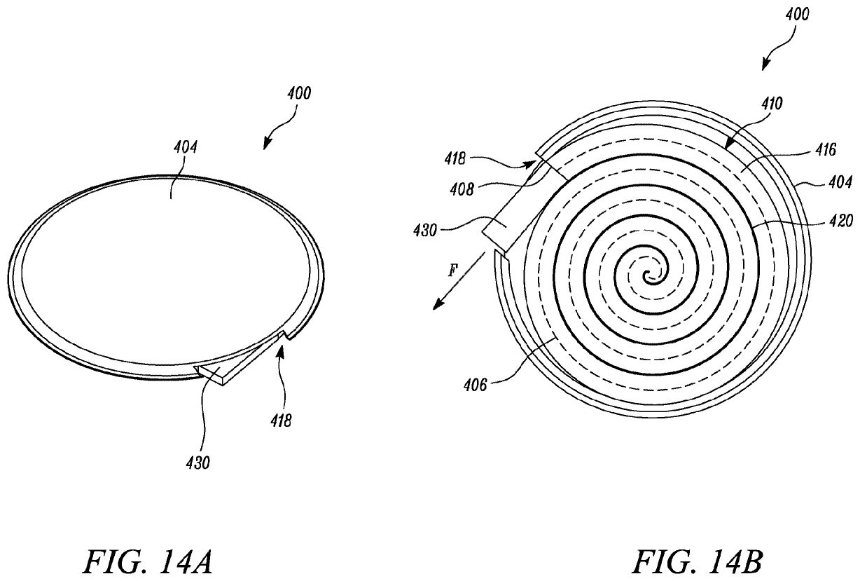

FIG. 14A is a schematic perspective view of an article 400. FIG. 14B is a schematic bottom view of the article 400. In some examples, the article 400 may be functionally equivalent to the article 100 of FIGS. 1A-3. Referring now to FIGS. 14A and 14B, the article 400 includes at least one substrate 404. In the illustrated embodiment of FIGS. 14A and 14B, the at least one substrate 404 is substantially circular in shape.

The article 400 further includes at least one adhesive member 410 (shown in FIG. 14B) connected to the at least one substrate 404. The at least one adhesive member 410 includes an adhesive layer 416 (shown in FIG. 14B) and at least one slit 420 (shown in FIG. 14B) extending through the at least one adhesive member 410. The article 400 further includes at least one tab 430 connected to at least the adhesive layer 416 of the at least one adhesive member 416 and extending from the at least one adhesive member 410. In some examples, the at least one substrate 404 includes at least one cutout 418 configured to at least partially receive the at least one tab 430 therethrough.

As shown in FIG. 14B, in some examples, the at least one slit 420 has a spiral shape, such that the at least one adhesive member 410 includes a spiral coil 406 corresponding to the spiral shape of the at least one slit 420. In some examples, the spiral coil 406 of the at least one adhesive member 410 includes an outer end 408. In some examples, the at least one tab 430 extends from the outer end 408 of the spiral coil 406.

The at least one tab 430 is configured to receive the pull force F from the user. Upon application of the pull force F on the at least one tab 430, the at least one adhesive member 410 at least partially separates adjacent to the at least one slit 420 and releases from each of the at least one substrate 404 and the external surface 102 (shown in FIG. 1A). Specifically, upon application of the pull force F on the at least one tab 430, the spiral coil 406 uncoils along the spiral shape of the at least one slit 420 and passes through the at least one cutout 418 of the at least one substrate 404, such that the at least one adhesive member 410 releases from each of the at least one substrate 404 and the external surface 102 (shown in FIG. 1A).

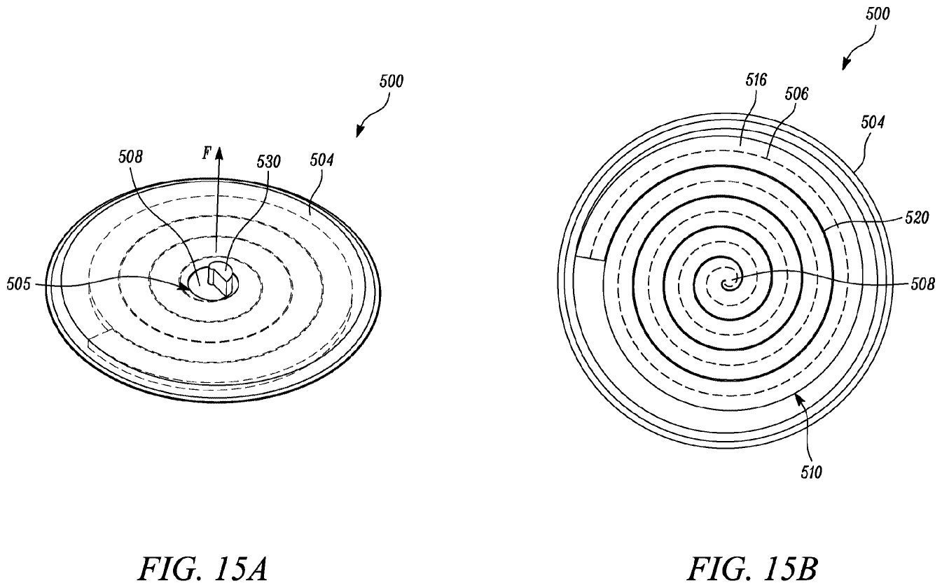

FIG. 15A is a schematic perspective view of an article 500. FIG. 15B is a schematic bottom view of the article 500. In some examples, the article 500 may be functionally equivalent to the article 100 of FIGS. 1A-3. Referring now to FIGS. 15A and 15B, the article 500 includes at least one substrate 504. In the illustrated embodiment of FIGS. 15A and 15B, the at least one substrate 404 is substantially circular in shape.

As shown in FIG. 15B, the article 500 further includes at least one adhesive member 510 connected to the at least one substrate 504. The at least one adhesive member 510 includes an adhesive layer 516 and at least one slit 520 extending through the at least one adhesive member 510. The article 500 further includes at least one tab 530 connected to at least the adhesive layer 516 of the at least one adhesive member 516 and extending from the at least one adhesive member 510.

In some examples, the at least one slit 520 has a spiral shape, such that the at least one adhesive member 510 includes a spiral coil 506 corresponding to the spiral shape of the at least one slit 520. In some examples, the spiral coil 506 of the at least one adhesive member 510 includes a central portion 508. In some examples, the at least one substrate 504 includes an aperture 505 (shown in FIG. 15A) extending therethrough and aligned with the central portion 508 of the spiral coil 506. In some examples, the at least one tab 530 extends from the central portion 508 of the spiral coil 506 through the aperture 505 of the at least one substrate 504.

The at least one tab 530 is configured to receive the pull force F from the user. Upon application of the pull force F on the at least one tab 530, the at least one adhesive member 510 at least partially separates adjacent to the at least one slit 520 and releases from each of the at least one substrate 504 and the external surface 102 (shown in FIG. 1A). Specifically, upon application of the pull force F on the at least one tab 530, the spiral coil 506 uncoils along the spiral shape of the at least one slit 520 and passes through the aperture 505 of the at least one substrate 504, such that the at least one adhesive member 510 releases from each of the at least one substrate 504 and the external surface 102 (shown in FIG. 1A).

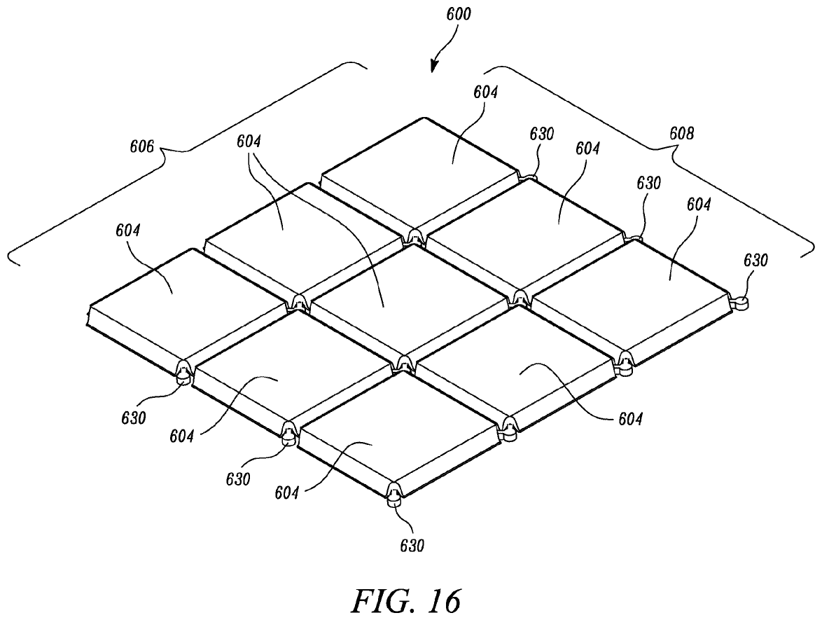

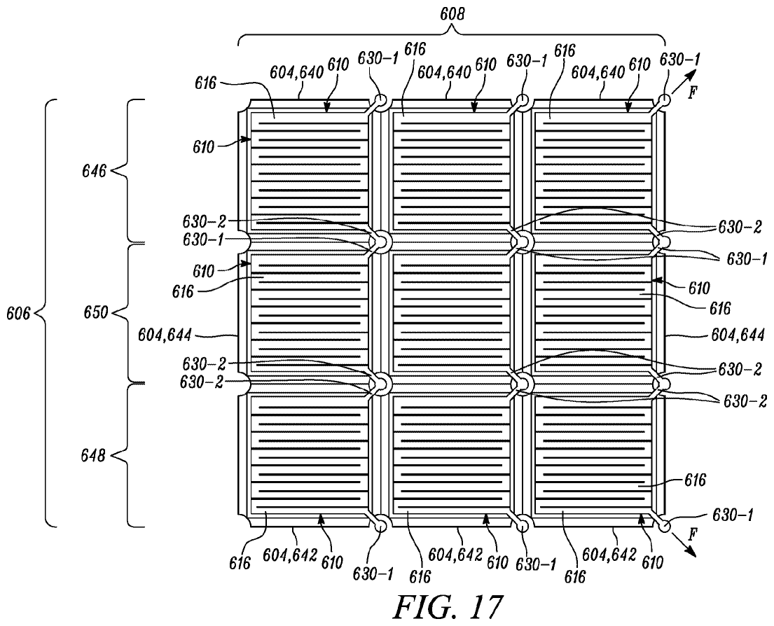

FIG. 16 is a schematic perspective view of an article 600. In some examples, the article 600 may be functionally equivalent to the article 100 of FIGS. 1A-3. FIG. 17 is a schematic bottom view of the article 600. Referring now to FIGS. 16 and 17, the article 600 includes at least one substrate 604. In some examples, the at least one substrate 604 includes a plurality of substrates 604 connected to each other. In some examples, the plurality of substrates 604 are arranged in a rectangular array comprising three or more rows 606 and one or more columns 608.

As shown in FIG. 17, in some examples, the plurality of substrates 604 includes a plurality of first end substrates 640 arranged in a first end row 646 from the plurality of rows 606, a plurality of second end substrates 642 arranged in a second end row 648 from the plurality of rows 606, and one or more intermediate substrates 644 arranged in one or more intermediate rows 650 from the plurality of rows 606. In some examples, the one or more intermediate rows 650 are disposed between the first end row 646 and the second end row 648.

The article 600 further includes at least one adhesive member 610 connected to the at least one substrate 604. In some examples, the at least one adhesive member 610 includes a plurality of adhesive members 610 corresponding to the plurality of substrates 604. In some examples, each adhesive member 610 from the plurality of adhesive members 610 is connected to a corresponding substrate 604 from the plurality of substrates 604. In some examples, each adhesive member 610 from the plurality of adhesive members 610 is connected to an adjacent adhesive member 610 from the plurality of adhesive members 610. Each of the plurality of adhesive members 610 includes an adhesive layer 616.

The article 600 further includes at least one tab 630 connected to at least the adhesive layer 616 of the at least one adhesive member 616 and extending from the at least one adhesive member 610. In some examples, the at least one tab 630 includes a pair of tabs 630-1, 630-2 extending from a corresponding adhesive member 610 from the plurality of adhesive member 610 and spaced apart from each other. In some examples, at least one tab 630-1, 630-2 from the pair of tabs 630-1, 630-2 is connected to an adjacent tab 630-1, 630-2 from the pair of tabs 630-1, 630-2 extending from the adjacent adhesive member 610, thereby connecting the corresponding adhesive member 610 to the adjacent adhesive member 610.

In some examples, for each of the first end substrates 640 and the second end substrates 642 arranged in a corresponding column 608 from the one or more columns 608, one tab 630-1 from the pair of tabs 630-1, 630-2 is configured to receive the pull force F from the user. In some examples, for each of the first end substrates 640 and the second end substrates 642 arranged in a corresponding column 608 from the one or more columns 608, the other tab 630-2 from the pair of tabs 630-1, 630-2 is connected to the adjacent tab 630-1, 630-2 extending from the corresponding adhesive member 610 of the adjacent intermediate substrate 604 arranged in the corresponding column 608. In some examples, for each of the intermediate substrates 650 arranged in a corresponding column 608 from the one or more columns 608, the pair of tabs 630-1, 630-2 are attached to the corresponding adjacent tabs 630-1, 630-2 extending from the corresponding adhesive members 610 of the opposing substrates 604 arranged in the corresponding column 608.

Specifically, for each first end substrate 640 arranged in a corresponding column 608, the tab 630-2 is connected to the adjacent tab 630-1 extending from the corresponding adhesive member 610 of the adjacent intermediate substrate 604 arranged in the corresponding column 608. Further, for each second end substrate 642 arranged in a corresponding column 608, the tab 630-2 is connected to the adjacent tab 630-2 extending from the corresponding adhesive member 610 of the adjacent intermediate substrate 604 arranged in the corresponding column 608.

Moreover, for each first end substrate 640 arranged in a corresponding column 608, the tab 630-1 is configured to receive the pull force F from the user. Similarly, for each second end substrate 642 arranged in a corresponding column 608, the tab 630-1 is configured to receive the pull force F from the user. In some examples, upon application of the pull force F on the one tab 630-1 from the pair of tabs 630-1, 630-2, the plurality of adhesive members 610 arranged in a corresponding column 608 from the one or more columns 608 are released successively from the external surface 102 (shown in FIG. 1A) and the corresponding substrates 604.

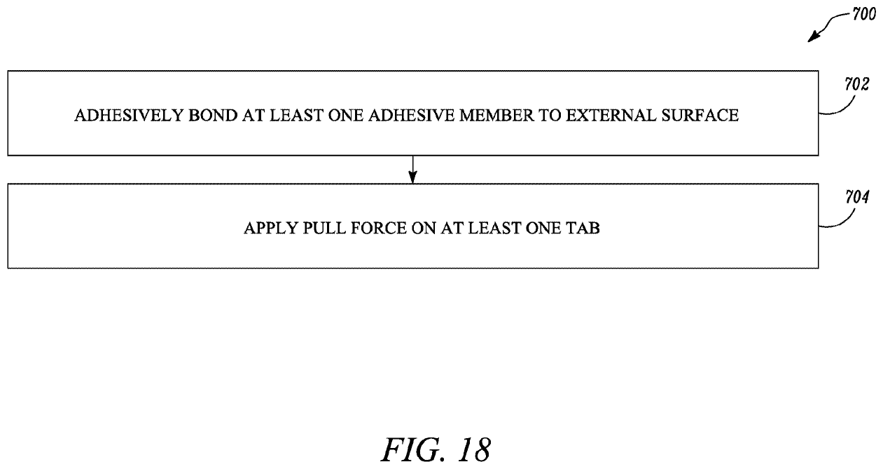

FIG. 18 is a flow chart illustrating a method 700 of using the article 100, 400, 500, 600 article having at least one substrate 104, 404, 504, 604, at least one adhesive member 110, 210, 310, 410, 510, 610 connected to the at least one substrate 104, 404, 504, 604, and at least one tab 130, 230, 330, 430, 530, 630 connected to the at least one adhesive member 110, 210, 310, 410, 510, 610. The method 700 will be described with reference to the article 100, 400, 500, 600 of FIGS. 1A-17.

Referring now to FIGS. 1A-18, at step 702, the method 700 includes adhesively bonding the at least one adhesive member 110, 210, 310, 410, 510, 610 to the external surface 102. Referring now to FIGS. 1A-18, at step 704, the method 700 further includes applying the pull force F on the at least one tab 130, 230, 330, 430, 530, 630 to cause at least a partial separation of the at least one adhesive member 110, 210, 310, 410, 510, 610 adjacent to the at least one slit 120, 220, 320, 420, 520, thereby releasing the at least one adhesive member 110, 210, 310, 410, 510, 610 from each of the at least one substrate 104, 404, 504, 604 and the external surface 102.

In some examples, the at least one adhesive member 110, 210, 310, 410, 510, 610 releases from each of the at least one substrate 104, 404, 504, 604 and the external surface 102 by at least one of necking and rolling.

In some examples, the at least one adhesive member 610 includes a plurality of adhesive members 610 connected to each other. In some examples, applying the pull force F on the at least one tab 630 connected to one of the plurality of adhesive members 610 causes the plurality of adhesive members 610 to successively release from each of the at least one substrate 604 and the external surface 102.

In some examples, the at least one slit 420, 520 has a spiral shape, such that the at least one adhesive member 410, 510 includes the spiral coil 406, 506 corresponding to the spiral shape of the at least one slit 420, 520. In some examples, upon application of the pull force F, the spiral coil 406, 506 of the at least one adhesive member 410, 510 uncoils along the spiral shape of the at least one slit 420, 520.

In some examples, the at least one slit 120 has a plurality of slits 120 spaced apart from each other and forming the serpentine pattern 140 of the at least one adhesive member 110. In some examples, upon application of the pull force F, the serpentine pattern 140 of the at least one adhesive member 110 unwinds along the plurality of slits 120.

In some examples, the method 700 further includes removing the release liner 106 from the at least one adhesive member 110, 210, 310, 410, 510, 610 prior to bonding the at least one adhesive member 110, 210, 310, 410, 510, 610 to the external surface 102.

In the article 100, 400, 500, 600 of the present disclosure, the at least one tab 130, 230, 330, 430, 530, 630 is connected to at least the adhesive layer 116, 216, 316, 416, 516, 616 of the at least one adhesive member 110, 210, 310, 410, 510, 610. The at least one tab 130, 230, 330, 430, 530, 630 may be accessible to the user of the article 100, 400, 500, 600. Pulling the at least one tab 130, 230, 330, 430, 530, 630 may cause at least a partial separation of the at least one adhesive member 110, 210, 310, 410, 510, 610 adjacent to the at least one slit 120, 220, 320, 420, 520, thereby causing the at least one adhesive member 110, 210, 310, 410, 510, 610 to release from each of the at least one substrate 104, 404, 504, 604 and the external surface 102. Thus, the user may be able to easily remove the article 100, 400, 500, 600 from the external surface 102.

The at least one adhesive member 110, 210, 310, 410, 510, 610 may include a bulk material allowing the at least one adhesive member 110, 210, 310, 410, 510, 610 to at least partially separate adjacent to the at least one slit 120, 220, 320, 420, 520 when the at least one tab 130, 230, 330, 430, 530, 630 is pulled. Further, the at least one slit 120, 220, 320, 420, 520 may allow reduction in a force required to release the at least one adhesive member 110, 210, 310, 410, 510, 610 (e.g., through necking) as compared to conventional adhesive members that include a continuous bulk of adhesive.

The at least one slit 120, 220, 320, 420, 520 of the present disclosure may be continuous and may extend from the at least one tab 130, 230, 330, 430, 530, 630 until an end of the at least one adhesive member 110, 210, 310, 410, 510, 610 along the first major surface 112 or the second major surface 114. Thus, the at least one slit 120, 220, 320, 420, 520 may allow the at least one adhesive member 110, 210, 310, 410, 510, 610 to be formed as a single, elongated continuous bulk material with a narrow cross-sectional area. This may enable a smaller necking cross-sectional area for the at least one adhesive member 110, 210, 310, 410, 510, 610 upon application of the pull force F on the at least one tab 130, 230, 330, 430, 530, 630, thereby reducing a magnitude of the pull force F required for releasing the at least one adhesive member 110, 210, 310, 410, 510, 610. Therefore, an impact of releasing the at least one adhesive member 110, 210, 310, 410, 510, 610 on both the at least one substrate 104, 404, 504, 604, and the external surface 102 may be minimized.

In some examples, the at least one slit 120, 220, 320, 420, 520 includes a plurality of slits 220, 320 that are substantially parallel to each other. This may divide a necking region of the at least one adhesive member 210, 310 into a plurality of smaller necking regions of reduced cross-sectional area. Therefore, the at least one adhesive member 210, 310 of the present disclosure may further reduce the magnitude of the pull force F required for releasing the at least one adhesive member 210, 310.

In some examples, the plurality of smaller necking regions with reduced cross-sectional area generated in the at least one adhesive member 210, 310 through the plurality of slits 220, 320 may be activated or stretched individually, sequentially, or in parallel, upon application of the pull force F on the at least one tab 230, 330. Further, in some examples, the at least one tab 230, 330 may include one or more tabs attached to each of the plurality of adhesive regions 256 to aid in removal of the at least one adhesive member 210, 310. In some examples, the plurality of smaller necking regions with reduced cross-sectional area may result in a lower pull force F being required to remove the at least one adhesive member 110, 210, 310, 410, 510, 610 from each of the at least one substrate 104, 404, 504, 604 and the external surface 102.

It should be noted that the at least one slit 120, 220, 320, 420, 520 of the at least one adhesive member 110, 210, 310, 410, 510, 610 may not significantly reduce adhesion of the at least one adhesive member 110, 210, 310, 410, 510, 610 to the external surface 102 (such as the skin of a user) while facilitating release of the at least one adhesive member 110, 210, 310, 410, 510, 610 from each of the at least one substrate 104, 404, 504, 604 and the external surface 102 without any significant damage or residue. Moreover, the at least one slit 120, 220, 320, 420, 520 may allow transmission of moisture through the at least one adhesive member 110, 210, 310, 410, 510, 610 in case where the external surface 102 is the skin of the user, thereby improving a permeability and a breathability of the at least one adhesive member 110, 210, 310, 410, 510, 610.

In the present detailed description of the preferred embodiments, reference is made to the accompanying drawings, which illustrate specific embodiments in which the invention may be practiced. The illustrated embodiments are not intended to be exhaustive of all embodiments according to the invention. It is to be understood that other embodiments may be utilized, and structural or logical changes may be made without departing from the scope of the present invention. The following detailed description, therefore, is not to be taken in a limiting sense, and the scope of the present invention is defined by the appended claims.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components, or layers for example.

Various examples have been described. These and other examples are within the scope of the following claims.

1. An article comprising:
at least one substrate;
at least one adhesive member comprising a first major surface and a second major surface opposite to the first major surface, wherein the first major surface is connected to the at least one substrate, the at least one adhesive member further comprising:
an adhesive layer forming the second major surface of the at least one adhesive member, the adhesive layer comprising an adhesive configured to bond the at least one adhesive member to an external surface; and
at least one slit extending through the at least one adhesive member from the first major surface to the second major surface, wherein the at least one slit has a spiral shape, such that the at least one adhesive member comprises a spiral coil corresponding to the spiral shape of the at least one slit; and
at least one tab connected to at least the adhesive layer of the at least one adhesive member and extending from the at least one adhesive member, wherein the at least one tab is configured to receive a pull force from a user, and wherein, upon application of the pull force on the at least one tab, the at least one adhesive member at least partially separates adjacent to the at least one slit and releases from each of the at least one substrate and the external surface.

2.-13. (canceled)

14. The article of claim 1, wherein the at least one tab tapers away from the at least one adhesive member and is D-shaped.

15.-18. (canceled)

19. The article of claim 1, wherein the at least one tab comprises a curved portion extending from the at least one adhesive member and a disc-shaped portion connected to the curved portion distal to the at least one adhesive member.

20. The article of claim 1, wherein the at least one tab comprises a linear portion extending from the at least one adhesive member and a disc-shaped portion connected to the linear portion distal to the at least one adhesive member.

21. (canceled)

22. The article of claim 1, wherein the spiral coil of the at least one adhesive member comprising an outer end, and wherein the at least one tab extends from the outer end of the spiral coil.

23. The article of claim 1, wherein the spiral coil of the at least one adhesive member comprises a central portion, wherein the at least one substrate comprises an aperture extending therethrough and aligned with the central portion of the spiral coil, and wherein the at least one tab extends from the central portion of the spiral coil through the aperture of the at least one substrate.

24. The article of claim 1, wherein the at least one substrate comprises at least one cutout configured to at least partially receive the at least one tab therethrough.

25. The article of claim 1, wherein the at least one adhesive member further comprises:
a core layer comprising a first core major surface facing the at least one substate and a second core major surface opposing the first core major surface, wherein the adhesive layer is disposed on the second core major surface; and
another adhesive layer disposed on the first core major surface and bonding the at least one adhesive member to the at least one substrate.

26. The article of claim 25, wherein the core layer comprises a foam.

27. The article of claim 25, wherein the at least one tab is further connected to the core layer and the another adhesive layer.

28. The article of claim 1, wherein the at least one tab and the at least one adhesive member have the same layered construction.

29. The article of claim 1, wherein the adhesive layer further forms the first major surface of the at least one adhesive member and is bonded to the at least one substrate, and wherein the adhesive layer further comprises a plurality of strengthening elements embedded within the adhesive.

30. The article of claim 29, wherein the plurality of strengthening elements comprise at least one of cylindrical members and flat members.

31. The article of claim 1, further comprising a release liner releasably attached to the adhesive layer, wherein the release liner is removed prior to bonding of the adhesive layer with the external surface.

32. The article of claim 1, wherein the adhesive is a pressure sensitive adhesive or a stretch-release adhesive.

33. The article of claim 1, wherein the at least one substrate comprises a main portion connected with the at least one adhesive member, and wherein the main portion is one of polygonal, circular, oval, and elliptical.

34.-37. (canceled)

38. The article of claim 1, wherein the at least one tab is adhesive-free.

39. The article of claim 1, wherein the at least one tab comprises an adhesive layer.

40. The article of claim 1, wherein the at least one adhesive member comprises a plurality of adhesive regions disposed adjacent to the at least one slit, and wherein each of the plurality of adhesive regions has a cross-sectional area less than a total cross-sectional area of the at least one adhesive member.

41. A method of using an article having at least one substrate, at least one adhesive member connected to the at least one substrate, and at least one tab connected to the at least one adhesive member, the method comprising:
adhesively bonding the at least one adhesive member to an external surface; and
applying a pull force on the at least one tab to cause at least a partial separation of the at least one adhesive member adjacent to at least one slit, thereby releasing the at least one adhesive member from each of the at least one substrate and the external surface,
wherein the at least one slit has a spiral shape, such that the at least one adhesive member comprises a spiral coil corresponding to the spiral shape of the at least one slit, and wherein, upon application of the pull force, the spiral coil of the at least one adhesive member uncoils along the spiral shape of the at least one slit.

42. The method of claim 41, wherein the at least one adhesive member releases from each of the at least one substrate and the external surface by at least one of necking and rolling.

43.-45. (canceled)

46. The method of claim 41, further comprising removing a release liner from the at least one adhesive member prior to bonding the at least one adhesive member to the external surface.
